(19) United States
(12) Patent Application Publication
(10) Pub. No.: US 2023/0250124 A1
(43) Pub. Date: Aug. 10, 2023

(54) SUBSTANTIALLY PURE CLARITHROMYCIN 9-OXIME AND ITS PREPARATION THEREOF

(71) Applicants: HIKAL LIMITED, (US); GALAPAGOS, (US)

(72) Inventors: Sudhir NAMBIAR, Pune (IN); Rakesh Ramesh GANORKAR, Pune (IN); Kiran CHAUDHARI, Pune (IN); Anil GOHAR, Pune (IN); Deeksha SINGH, Pune (IN); Patrick CLAERHOUT STIJN POL, Mechelen (BE)

(73) Assignees: HIKAL LIMITED, Pune (IN); GALAPAGOS, Mechelen (BE)

(21) Appl. No.: 17/773,279

(22) PCT Filed: Oct. 27, 2020
(86) PCT No.: PCT/IB2020/060051

§ 371 Date: Apr. 29, 2022

(30) Foreign Application Priority Data
Oct. 29, 2019 (IN) 201921043722

(51) Int. Cl. C07H 17/08; C07H 1/00

(52) U.S. Cl. CPC C07H 17/08; C07H 1/00

(57) ABSTRACT

The present invention relates to substantially pure Clarithromycin 9-oxime more particularly Clarithromycin 9(E)-oxime having purity more than 98% and corresponding (Z)-isomer not more than 1%. The present invention further relates to a process for preparation of Clarithromycin 9(E)-oxime of formula (I), its pharmaceutically acceptable salts and purification.

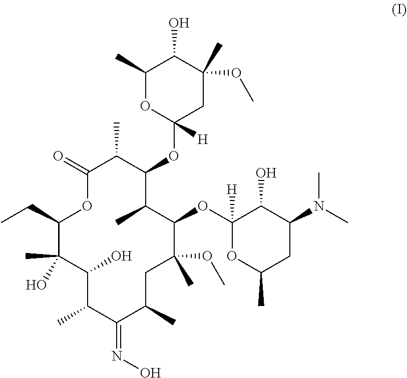 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)C)[C@H]2O)[C@](C)(OC)C[C@@H](C)/C(=N\O)[C@H](C)[C@@H](O)[C@]1(C)O]

### RELATED APPLICATION

This application claims the benefit to Indian Provisional Application No. IN201921043722 filed on Oct. 29, 2019, the contents of which are incorporated by reference herein.

### FIELD OF THE INVENTION

The present invention relates to substantially pure Clarithromycin 9-oxime more particularly Clarithromycin 9(E)-oxime having purity more than 98% and corresponding (Z)-isomer not more than 1%. The present invention further relates to a process for preparation of Clarithromycin 9(E)-oxime of formula (I), its pharmaceutically acceptable salts and purification.

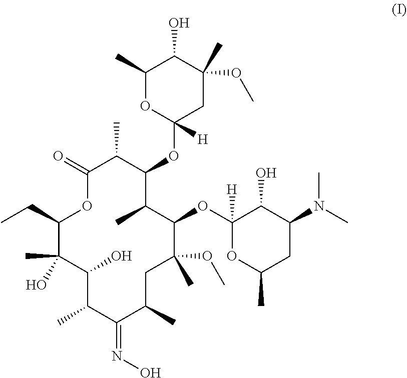 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)C)[C@H]2O)[C@](C)(OC)C[C@@H](C)/C(=N\O)[C@H](C)[C@@H](O)[C@]1(C)O]

### BACKGROUND OF THE INVENTION

Clarithromycin (6-O-methylerythromycin A) is a potent macrolide antibiotic used to treat various bacterial infections including pneumonia, Helicobacter pylori, and as an alternative to penicillin in strep throat. Clarithromycin is very effective against aerobic and anaerobic Gram-positive bacteria and Gram-negative bacteria. Clarithromycin 9-oxime (6-O-methylerythromycin A 9-oxime) of formula (III) is an advanced intermediate used in various APIs which are currently under pre-clinical studies and acts as an antibiotic with great antibacterial activity. Clarithromycin 9-oxime of formula (III) has molecular formula C₃₈H₇ON₂O₁₃ and molecular weight 762.9.

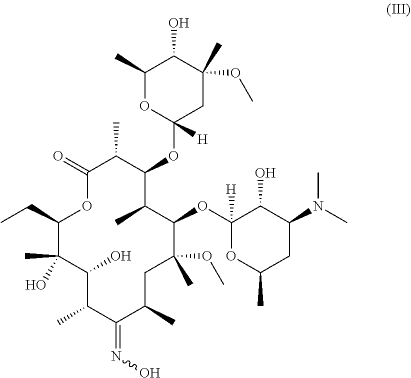 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)C)[C@H]2O)[C@](C)(OC)C[C@@H](C)C(=NO)[C@H](C)[C@@H](O)[C@]1(C)O]

Clarithromycin 9-oxime of formula (III) is known to exist in two forms namely, Clarithromycin 9(E)-oxime [9(E)-6-O-Methyl-erythromycin oxime] of formula (I) and Clarithromycin 9(Z)-oxime [9(Z)-6-O-Methyl-erythromycin oxime] of formula (II).

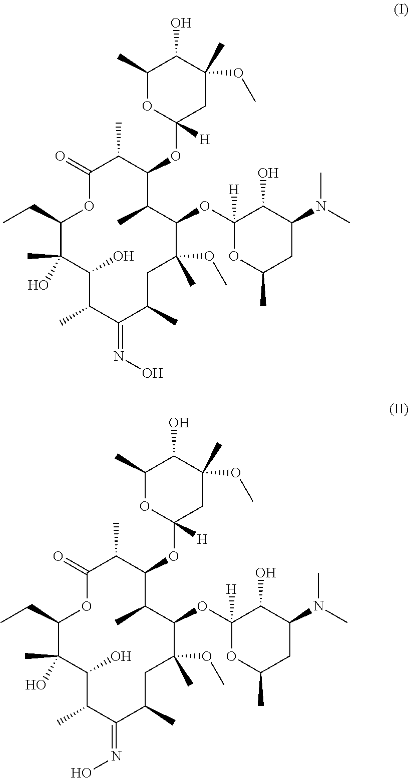

As both the isomers are structurally similar, it is difficult to obtain Clarithromycin 9(E)-oxime of formula (I) substantially free from Clarithromycin 9(Z)-oxime of formula (II).

Clarithromycin 9-oxime was first disclosed in U.S. Pat. No. 4,680,386 A (hereinafter US′386) as a 6-O-methylerythromycin A derivative. US′386 describes a process for the preparation of 6-O-methylerythromycin A 9-oxime. However, it does not describe about the purity and content of corresponding E or Z-oxime.

U.S. Pat. No. 5,837,829 A (hereinafter US′829) describes a process of preparation of a 6-O-methylerythromycin A-9-oxime from 2′,4″-O-bis(trimethylsilyl)-6-O-methylerythromycin A 9(0-t-butyldiphenylsilyl) oxime. The major drawback is that the process involves expensive silylating agent which is sensitive towards acids and bases. Further, US′829 does not describe the method of separation of isomers.

U.S. Pat. No. 6,110,965 A describes a process of preparation of a 6-O-methylerythromycinA-9(E)-oxime from a 6-O-methylerythromycin A. This process involves separation of the 6-O-methylerythromycin A 9 (E)-oxime and 6-O-methylerythromycin A 9 (Z)-oxime by column chromatography.

The PCT patent application no. WO2009/007988 A1, describes a process of preparation of a 6-O-methylerythromycin A 9 (E)-oxime from the 6-O-methylerythromycin A. This process does not describe the content of an undesired isomer (Z-isomer) of 6-O-methylerythromycin A 9-oxime and a purity of desired isomer is only 95%.

The references discussed above disclose a preparation of Clarithromycin oxime from erythromycin in any order of sequence i.e., methylation of 6-OH followed by oxime formation or oxime formation followed by methylation. These processes involve protection/deprotection steps, low purity of oxime and involve tedious purification process. Thus, achieving pure Clarithromycin 9(E)-oxime remains a need, therefore, the present inventors have come-up with an improved process to get substantially pure Clarithromycin 9(E)-oxime with or without purification.

### OBJECTIVES OF THE INVENTION

The main object of the present invention is to provide a process for the preparation of substantially pure a Clarithromycin 9-oxime of formula (III).

Another object of the present invention is to provide a process for the preparation of substantially pure the Clarithromycin 9-oxime of formula (III) with or without involving purification.

Yet another object of the present invention is to provide a process for the preparation of substantially pure a Clarithromycin 9(E)-oxime of formula (I), substantially free from corresponding a 9(Z)-oxime of formula (II).

### SUMMARY OF THE INVENTION

In one aspect, the present invention provides a substantially pure Clarithromycin 9(E)-oxime of formula (I) and its pharmaceutically acceptable salts thereof, having purity greater than 98%, whereas the corresponding Z-isomer is not more than 1%.

In another aspect, the present invention provides a process for the preparation of substantially pure Clarithromycin 9(E)-oxime of formula (I) and its pharmaceutically acceptable salts thereof, having purity greater than 98%, whereas the corresponding Z-isomer is not more than 1%.

In another aspect, the present invention provides a process for the preparation of substantially pure Clarithromycin 9-oxime of formula (III) and its salts thereof.

In another aspect, the present invention provides a process for the preparation of substantially pure the Clarithromycin 9-oxime of formula (III) and its salts thereof with or without involving purification.

In another aspect, the present invention provides a process for the preparation of substantially pure the Clarithromycin 9(E)-oxime of formula (I) and its salts thereof.

In another aspect, the present invention provides a process for the preparation of substantially pure the Clarithromycin 9(E)-oxime of formula (I) with or without involving purification.

In one aspect, the present invention provides substantially pure Clarithromycin 9(E)-oxime of formula (I) and its pharmaceutically acceptable salts thereof, having purity greater than 98% where the corresponding Clarithromycin 9(Z)-oxime is not more than 1% which is obtained by a process comprising the steps:

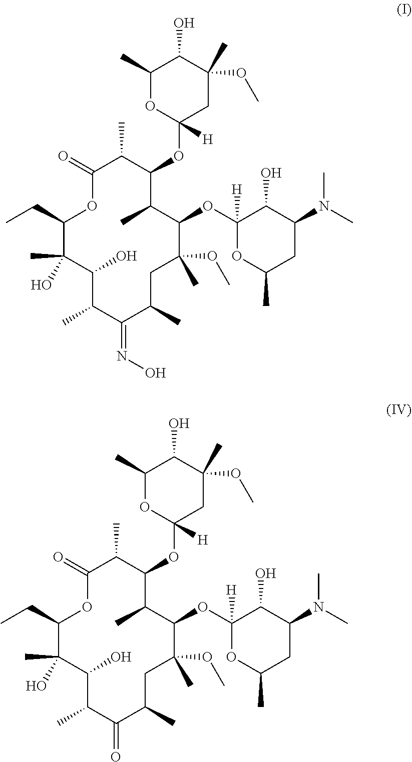

a) reacting Clarithromycin of formula (IV) with a hydroxylamine hydrochloride in presence of a base and a solvent;
b) adding solvent(s) to the concentrated reaction solution, adjusting pH of the organic layer and concentrating the organic layer;
c) adding a chlorinated solvent to a concentrated reaction mass and allowed to separate Z-isomer;
d) purifying the Clarithromycin 9(E)-oxime of formula (I).

In one embodiment, the present invention provides a process for the preparation of substantially pure Clarithromycin 9-oxime of formula (III) comprising the steps:
a) reacting Clarithromycin of formula (IV) with a hydroxylamine hydrochloride in presence of a base and a solvent;
b) adding solvent(s) to a concentrated reaction solution, adjusting pH of the organic layer and concentrating the organic layer;
c) optionally purifying the Clarithromycin 9-oxime of formula (III).

In another embodiment, the present invention provides a process for the preparation of substantially pure Clarithromycin 9(E)-oxime of formula (I) which comprising the steps:
a) reacting Clarithromycin of formula (IV) with a hydroxylamine hydrochloride in presence of a base and a solvent;
b) adding solvent(s) to a concentrated reaction solution, adjusting pH of organic layer and concentrating the organic layer;
c) adding a chlorinated solvent to concentrated reaction mass and allowed to separate Z-isomer.

In one embodiment, the substantially pure Clarithromycin 9(E)-oxime contains more than 90% E isomer.

In one embodiment, the substantially pure Clarithromycin 9(E)-oxime contains more than 95% E isomer.

In one embodiment, the substantially pure Clarithromycin 9(E)-oxime contains more than 98% E isomer.

In another embodiment, the present invention provides a process for the preparation of substantially pure Clarithromycin 9(E)-oxime of formula (I) which comprising the steps:
a) reacting Clarithromycin of formula (IV) with hydroxylamine hydrochloride in presence of a base and a solvent;
b) adding solvent(s) to the concentrated reaction solution, adjusting pH of organic layer and concentrated the organic layer;
c) adding a chlorinated solvent to concentrated reaction mass and allowed to separate Z-isomer;
d) purifying the Clarithromycin 9(E)-oxime of formula (I).

In one embodiment, the substantially pure Clarithromycin 9(E)-oxime contains more than 95% E isomer.

In one embodiment, the substantially pure Clarithromycin 9(E)-oxime contains more than 98% E isomer.

For the purpose of this specification, the meaning of term “Clarithromycin of formula (IV)” as used hereinabove includes Clarithromycin of formula (IV) in any polymorphic form, or hydrate, clathrate, solvate or their mixtures and in any state of purity, unless specifically mentioned.

For the purpose of this specification, the meaning of term “substantially pure” Clarithromycin 9-oxime of formula (III) herein contains not more than 10% of other impurities; preferably not more than 5%.

For the purpose of this specification, the meaning of term “substantially pure” Clarithromycin 9(E)-oxime of formula (I) herein contains not more than 5% of Z-isomer; preferably not more than 2%; more preferably not more than 1% of Z-isomer.

### DETAILED DESCRIPTION OF THE INVENTION

The present invention now will be described more fully hereinafter. The invention is embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly indicates otherwise.

In an embodiment of the present invention, the term “salt” means pharmacologically acceptable salts with a organic acids such as acetic acid, propionic acids, butyric acid, trifluoroacetic acid, maleic acid, tartaric acid, citric acid, stearic acid, succinic acid, ethyl succinic acid, methane sulfonic acid, benzenesulfonic acid, p-toluene sulfonic acid, lauryl sulfonic acid, malic acid, aspartic acid, glutamic acid and the like; and inorganic acids such as hydrochloric acid, sulfonic acid, phosphoric acid, hydroiodic acid and the like.

The term solvent used herein, refers to the single solvent or mixture of solvents.

The term “concentrated” as used herein in the specification refers to removal of solvent to minimum stirrable volume.

For the purpose of the specification, the meaning of term “substantially free” as used hereinabove refers to Clarithromycin 9(E)-oxime of formula (I) containing not more than 1.0% Clarithromycin 9(Z)-oxime of formula (II).

The “purification” step mentioned herein specification is carried out by using different purification techniques well known in prior art.

The process is illustrated in the following general synthetic scheme:

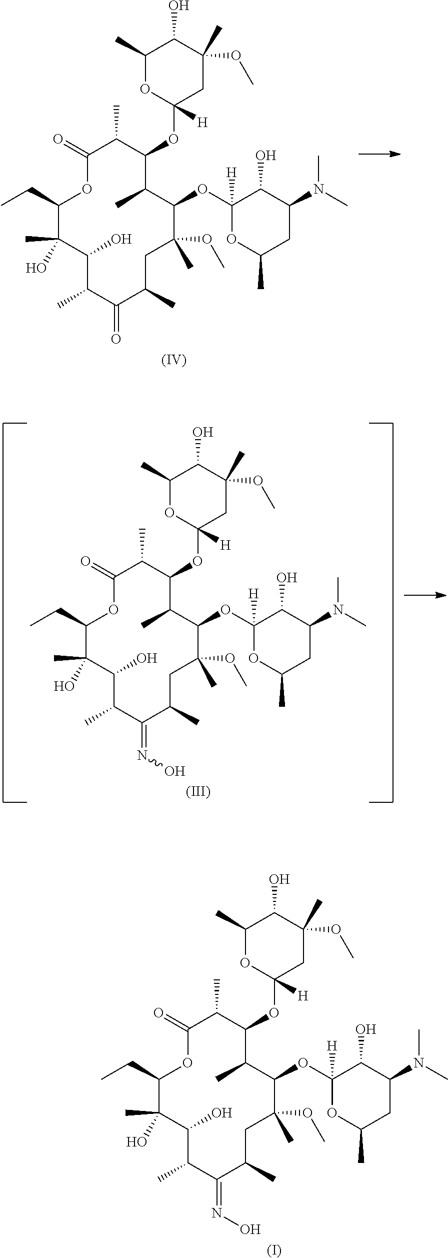

In another embodiment of the present invention, wherein the base used in step (a) is selected from group consisting of mono, di and tri alkyl amine such as triethyl amine, N,N-diisopropylethylamine, 1,8 diazabicyclo[5.4.0]undec-7-ene, 1,5-diazabicyclo[4.3.0]non-5-ene, 1,5-diazabicyclo[4.3.0]non-5-ene, imidazole, 4-dimethylaminopyridine, potassium carbonate, sodium carbonate, sodium bicarbonate, potassium bicarbonate, sodium hydroxide, potassium hydroxide, sodium methoxide, potassium methoxide, sodium ethoxide, sodium acetate trihydrate, sodium acetate, potassium acetate and the like.

In another embodiment of the present invention, wherein the solvent in step (a) and step (b) is selected from water, alcoholic solvent, chlorinated solvent, ethereal solvent, and the like or mixture thereof.

In another embodiment of the present invention, wherein the purification in step (d) is carried out in a solvent selected from water, alcoholic solvent, hydrocarbon solvent and ethereal solvent, polar protic or aprotic solvents and the like and mixture thereof.

In another embodiment of the present invention, wherein the alcoholic solvent in step (a), step (b) and step (d) is preferably selected from the group consisting of methanol, ethanol, isopropyl alcohol, n-propanol, n-butanol and the like or mixture thereof.

In another embodiment of the present invention, wherein the chlorinated solvent in step (a), step (b) and step (c) is preferably selected from the group consisting of dichloromethane, chloroform, ethylene dichloride, trichloroethylene, perchloroethylene and the like and mixture thereof.

In another embodiment of the present invention, wherein the ethereal solvent in step (a), step (b) and step (d) is preferably selected from the group consisting of tetrahydrofuran, diethyl ether, methoxyethane, dimethoxymethane, methyl tert-butyl ether, polyethylene glycol and the like and mixture thereof.

In another embodiment of the present invention, wherein the pH in step (b) is adjusted by using a base selected from the group consisting of potassium carbonate, sodium carbonate, sodium bicarbonate, potassium bicarbonate, sodium hydroxide, potassium hydroxide, sodium methoxide, potassium methoxide, sodium ethoxide and the like or mixture thereof.

In another embodiment of the present invention, wherein hydrocarbon solvent in step (d) is selected from aliphatic or aromatic hydrocarbon solvents such as toluene, xylene, cyclohexane, heptane, hexane, methylcyclohexane, petroleum ether and the like and mixture thereof.

In another embodiment of the present invention, wherein pH of reaction in step (a) is optional, it ranges from 6-10; preferably 7-8, where the pH is adjusted using sodium bicarbonate.

In another embodiment of the present invention, wherein the pH range in step (b) is between 6-10; preferably 7-8.

In yet another embodiment of the present invention, wherein the reaction, isolation and purification step are carried out between temperature 0° C. to 120° C.

The process of the present invention is described by the following example, which is illustrative only and should not be construed to limit the scope of the invention in any manner.

### EXPERIMENTATION

### Example 1: Preparation of Clarithromycin 9(E)-oxime

To a solution of hydroxylamine hydrochloride (0.92 Kg) in methanol (2.5 L), sodium acetate trihydrate (1.85 Kg) was added at room temperature (rt) followed by addition of Clarithromycin (0.5 Kg). The reaction mixture was refluxed under stirring for 20 to 24 h. The reaction solution was concentrated to minimum stirrable volume and dichloromethane (3.0 L) and water (2.5 L) was charged. The organic layer was separated and treated with 10% sodium bicarbonate to adjust the pH about 8 and filtrate was extracted by dichloromethane, washed with brine and concentrated under vacuum. Dichloromethane was added to the concentrated mass, stirred for 30 min. at rt, filtered and concentrated. Isopropyl alcohol (3.5 L) was added to the concentrated organic layer at 40° C. to 50° C. and the resulting mass concentrated to minimum stirrable volume and further heated to 75° C. to 85° C. for 0-2 h. The reaction mixture was cooled to 0° C. to 5° C. under stirring, filtered and obtained solid was washed with isopropyl alcohol and dried to afford Clarithromycin 9(E)-oxime (0.32 Kg, 62.72% yield) with HPLC purity 98.19% and Clarithromycin9(Z)-oxime 0.84%, LCMS:764 [M+H]⁺;

¹H NMR (CDCl₃, 400 MHz)δ:5.079-5.052 (d, 1H), 4.921-4.910 (d, 1H), 4.525 (br s, 1H), 4.413-4.395 (d, 1H), 4.010-3.979 (m, 1H), 3.736-3.715 (m, 3H), 3.623-3.605 (d, 1H), 3.458-3.449 (m, 1H), 3.298-3.277 (m, 4H), 3.226-3.183 (q, 1H), 3.064 (s, 3H), 3.007-2.982 (m, 1H), 2.856-2.834 (m, 1H), 2.543-2.525 (m, 1H), 2.461-2.396 (m, 1H), 2.357-2.319 (d, 1H), 2.270 (s, 6H), 1.914-1.879 (m, 2H), 1.660-1.629 (d, 1H), 1.571-1.375 (m, 7H), 1.280-1.264 (d, 3H), 1.217-1.160 (m, 12H), 1.097-1.086 (m, 6H), 1.044-1.025 (d, 3H), 0.959-0.942 (d, 3H), 0.817-0.780 (t, 3H);

¹³C NMR (CDCl₃, 400 MHz)δ: 175.71, 170.15, 102.77, 95.97, 80.47, 78.67, 78.36, 77.99, 76.82, 74.05, 72.67, 71.20, 70.24, 68.59, 65.57, 65.28, 64.36, 51.13, 49.43, 45.05, 40.30, 39.03, 37.37, 34.87, 32.70, 29.16, 25.32, 25.25, 21.45, 21.38, 21.11, 19.97, 18.63, 18.55, 16.01, 14.93, 10.56, 9.13;

FTIR spectrometer with a NXRFT Raman Module and the samples dispersed in KBr pellets with characteristic peaks values 3421.25, 2978.19, 2941.04, 2882.48, 2831.58, 2793.93, 1746.47, 1715.30, 1644.10, 1460.93, 1403.33, 1349.50, 1288.36, 1264.59, 1250.18, 1184.68 1169.86, 1111.79, 1075.25, 1011.72 and 955.84 cm⁻¹.

### Example 2: Preparation of Clarithromycin 9(E)-oxime

To a solution of hydroxylamine hydrochloride (0.92 Kg) in methanol (2.5 L), sodium acetate trihydrate (1.85 Kg) was added at rt followed by addition of sodium bicarbonate (0.028 Kg) and Clarithromycin (0.5 Kg) at 10° C. to 20° C. The reaction mixture was refluxed under stirring for 20-24 h. The reaction solution was concentrated to minimum stirrable volume and dichloromethane (5.0 L) and water (3.0 L) was charged. The organic layer was separated and treated with 10% aq. sodium bicarbonate solution to adjust the pH about 8. The biphasic reaction mixture containing emulsion was filtered and clear biphasic filtrate was separate out, washed with brine and concentrated under vacuum. Dichloromethane was added to the concentrated mass, stirred for 30 min. at room temperature, filtered and concentrated. Isopropyl alcohol (2.0 L) was added to the concentrated organic layer at 40° C. to 50° C. and the resulting mass concentrated to minimum stirrable volume and further heated to 80° C. to 85° C. for 0-2 h. The reaction mixture was cooled to 0° C. to 10° C. under stirring, filtered and obtained solid was washed with isopropyl alcohol and dried to afford Clarithromycin 9(E)-oxime (0.34 Kg, 66.67% yield) with HPLC purity 98.19% and Clarithromycin 9(Z)-oxime 0.84%, LCMS:764 [M+H]⁺.

1. A substantially pure Clarithromycin 9(E)-oxime of formula (I) and its pharmaceutically acceptable salts thereof, having purity greater than 98%, whereas the corresponding Z-isomer is not more than 1%.

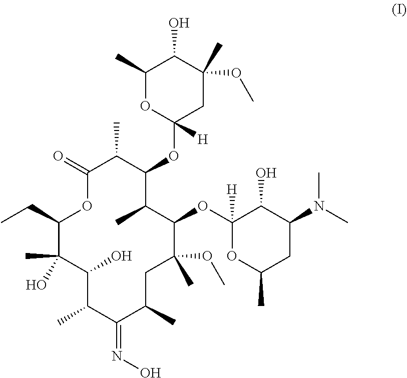 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)C)[C@H]2O)[C@](C)(OC)C[C@@H](C)/C(=N\O)[C@H](C)[C@@H](O)[C@]1(C)O]

2. The compound as claimed in claim 1, wherein the substantially pure Clarithromycin 9(E)-oxime of formula (I) and its pharmaceutically acceptable salts thereof, having purity greater than 98% where the corresponding Clarithromycin 9(Z)-oxime is not more than 1% which is obtained by a process comprising the steps: 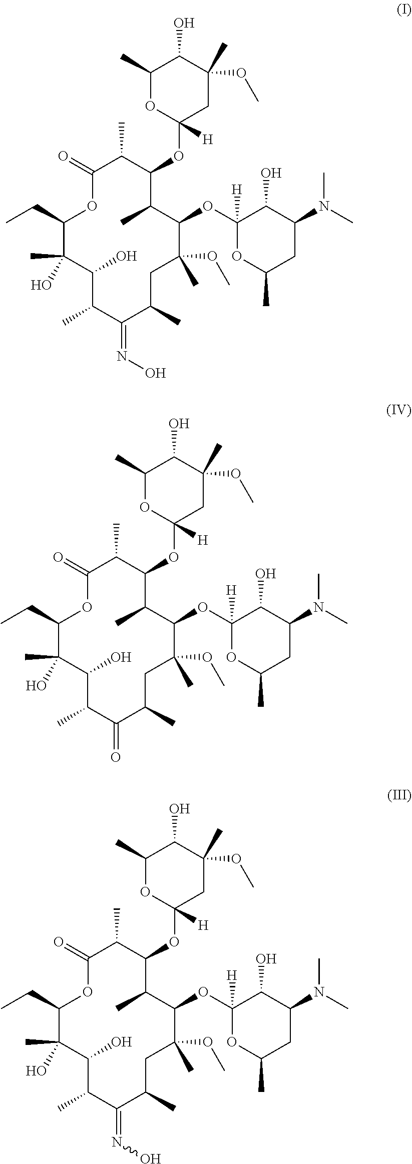
a) reacting Clarithromycin of formula (IV) with a hydroxylamine hydrochloride in presence of a base and a solvent;
b) adding solvent(s) to a concentrated reaction solution of step (a), adjusting pH of an organic layer and concentrating the organic layer to obtain a compound of formula (III), which is optionally purified;
c) adding a chlorinated solvent to a concentrated reaction mass and allowed to separate a Z-isomer;
d) optionally, purifying the Clarithromycin 9(E)-oxime of formula (I).

3. (canceled)

4. The process as claimed in claim 2 or 3, wherein the base in step (a) is selected from group consisting of mono, di and tri alkyl amine such as triethyl amine, N,N-diisopropylethylamine, 1,8 diazabicyclo[5.4.0]undec-7-ene, 1,5-diazabicyclo[4.3.0]non-5-ene, 1,5-diazabicyclo[4.3.0]non-5-ene, imidazole, 4-dimethylaminopyridine, potassium carbonate, sodium carbonate, sodium bicarbonate, potassium bicarbonate, sodium hydroxide, potassium hydroxide, sodium methoxide, potassium methoxide, sodium ethoxide, sodium acetate trihydrate, sodium acetate and potassium acetate.

5. The process as claimed in claim 2 or 3, wherein the solvent in step (a) and step (b) is selected from water, alcoholic solvent, chlorinated solvent, and ethereal solvent.

6. The process as claimed in claim 2 or 3, wherein the solvent used for purification is selected from water, alcoholic solvent, hydrocarbon solvent and ethereal solvent and polar protic or aprotic solvent.

7. The process as claimed in claim 5 or 6, wherein an alcoholic solvent is selected from the group consisting of methanol, ethanol, isopropyl alcohol-propanol and n-butanol.

8. The process as claimed in claim 5, wherein the chlorinated solvent is selected from the group consisting of dichloromethane, chloroform, ethylene dichloride, trichloroethylene and perchloroethylene.

9. The process as claimed in claim 5, wherein an ethereal solvent is selected from the group consisting of tetrahydrofuran, diethyl ether, methoxyethane, dimethoxymethane, methyl tert-butyl ether and polyethylene glycol.

10. The process as claimed in claim 2, wherein the pH is adjusted between 6 to 10 by using a base selected from the group consisting of potassium carbonate, sodium carbonate, sodium bicarbonate, potassium bicarbonate, sodium hydroxide, potassium hydroxide, sodium methoxide, potassium methoxide and sodium ethoxide.

11. (canceled)

12. The process as claimed in claim 6, wherein the hydrocarbon solvent is selected from aliphatic or aromatic hydrocarbon solvents such as toluene, xylene, cyclohexane, heptane, hexane, methylcyclohexane and petroleum ether.

13. The process as claimed in claim 2, wherein the substantially pure Clarithromycin 9(E)-oxime of formula (I) contains (E)-isomer more than 98% and (Z)-isomer not more than 1.0%.

14. The process as claimed in claim 2, wherein the Clarithromycin 9-oxime of formula (III) is substantially pure which contains not more than 5% of impurities.
